(19) United States
(12) Patent Application Publication
(10) Pub. No.: US 2016/0007993 A1
(43) Pub. Date: Jan. 14, 2016

(54) REVOLVING APPROXIMATION DEVICE

(71) Applicant: Boston Scientific Scimed, Inc., Maple Grove, MN (US)

(72) Inventors: Paul SMITH, Smithfield, RI (US); Samuel Raybin, Marlborough, MA (US); Naroun Suon, Lawrence, MA (US); Michelle Fater, Worcester, MA (US); Dylan Murphy, Walpole, MA (US); Robert B. DeVries, Northborough, MA (US)

(21) Appl. No.: 14/793,131

(22) Filed: Jul. 7, 2015

Related U.S. Application Data
(60) Provisional application No. 62/022,292, filed on Jul. 9, 2014.

(51) Int. Cl. A61B 17/062; A61B 17/06

(52) U.S. Cl. CPC A61B 17/0625; A61B 17/06066; A61B 2017/00862

(57) ABSTRACT

A device for treating tissue includes a helical needle extending about a longitudinal axis thereof from a distal tip to a proximal end and a control member extending longitudinally between a distal end releasably coupled to the proximal end of the helical needle and a proximal end which, when the helical needle is inserted into a body, remains outside the body, the control member configured such that rotation of the control member about the longitudinal axis rotates the helical needle to screw the helical needle distally into a target tissue.

### PRIORITY CLAIM

The present application claims priority to U.S. Provisional Patent Application Ser. No. 62/022,292 filed Jul. 9, 2014; the disclosure of which is incorporated herewith by reference.

### BACKGROUND

Physicians have become more willing to perform more aggressive interventional and therapeutic endoscopic procedures including, for example, removal of larger lesions (e.g., cancerous masses), tunneling under a mucosal layer of the gastro-intestional (GI) tract to treat tissues below the mucosa, full thickness removal of tissue, inserting devices through the GI tract and then penetrating the GI organ to treat tissue outside the GI tract, and endoscopic treatment/repair of post-surgical issues (e.g., post-surgical leaks, breakdown of surgical staple lines, anastomotic leaks). These procedures may increase the risk of perforating the wall of the GI tract, or may require closure of the GI tract wall as part of the procedure. Endoscopic closure reduces cost and may reduce patients' trauma, pain and inconvenience. However, conventional tissue closure devices may be insufficient to close certain perforations.

### SUMMARY

1. The present application, according to aspect 1, is directed to a device for treating tissue, comprising a helical needle extending about a longitudinal axis thereof from a distal tip to a proximal end and a control member extending longitudinally between a distal end releasably coupled to the proximal end of the helical needle and a proximal end which, when the helical needle is inserted into a body, remains outside the body, the control member configured such that rotation of the control member about the longitudinal axis rotates the helical needle to screw the helical needle distally into a target tissue.

2. The device of aspect 1, wherein a helical diameter of the helical needle varies along a length thereof.

3. The device of aspects 1 and 2, wherein the helical needle is formed of a superelastic shape memory material.

4. The device of aspects 1 to 3, wherein, when an unconstrained helical diameter of the helical needle is greater than an inner diameter of a working channel of an insertion instrument through which it is to be inserted to the target tissue within the body, the helical diameter is compressible via the working channel so that, when the helical needle is extended distally out of the working channel, the helical needle expands to the unconstrained helical diameter.

5. The device of aspects 1 to 4, wherein the proximal end of the control member is connected to a handle assembly including an actuator for rotating the control member.

6. The device aspects 1 to 5, wherein the control member and the helical needle are integrally formed.

7. The device of aspects 1 to 6, wherein the helical needle includes a lumen extending therethrough.

8. The device of aspect 7, further comprising a flexible suture slidably received within the lumen.

9. The device of aspect 8, wherein a distal end of the flexible suture includes a first anchoring element configured to lodge in the target tissue into which it is inserted.

10. The device of aspect 9, wherein the first anchoring element is one of T-shaped, a barb, a hook and a spiral.

11. The device of aspects 9 and 10, wherein the first anchoring element is deformable into an insertion configuration in which it extends substantially parallel to a longitudinal axis of the flexible suture while the flexible suture resides within the helical needle and, when the distal end of the flexible suture is extended out of the distal end of the helical needle, the first anchoring element moves under a natural bias to an anchoring position transverse to the longitudinal axis of the flexible suture.

12. The device of aspects 8 to 11, further comprising one of a locking element mountable over a portion of the flexible suture to lock the flexible suture within the body and a second anchoring element along a proximal portion of the flexible suture for anchoring the proximal portion of the flexible suture in a portion of the target tissue.

13. According to aspect 13, the present disclosure is also directed to a system for treating a tissue, comprising a needle extending along a helical path about a longitudinal axis, a control member extending longitudinally from a proximal end to a distal end releasably connected to the proximal end of the needle, and an actuator coupled to the proximal end of the control member such that rotation of the control member rotates the needle and screws the needle into a target tissue.

14. The system of aspect 13, further comprising a suture slidably received within a lumen of the needle, a distal end of the suture including an anchoring element so that when the distal end of the suture is moved distally past the distal end of the needle, the anchoring element becomes lodged in the target tissue into which it is inserted.

15. The system of aspects 13 and 14, wherein, when an unconstrained diameter of the helical path is greater than an inner diameter of a working channel through which it is to be inserted to the target tissue in the body, the needle is compressible via the working channel so that, when the needle is moved distally out of the working channel, the needle expands to the unconstrained diameter of the helical path.

The present disclosure is also directed to a method for treating a tissue opening, comprising inserting a helical needle through an insertion device to a tissue opening within a body, the helical needle extending along a helical path about a longitudinal axis to a tissue piercing distal end, advancing distally a control member, a distal end of which is coupled to a proximal end of the helical needle, to advance the helical needle out of the insertion device into a first portion of tissue at a first side of the tissue opening, the helical needle being screwed into the first portion of tissue by rotation of the control member about the longitudinal axis, moving the tissue opening toward a closed position by drawing the first portion of tissue toward a second side of the tissue opening opposite the first side of the tissue opening using the insertion device, and rotating the control member to screw the helical needle into a second portion of tissue along the second side of the tissue opening to hold the tissue opening in the closed position.

### BRIEF DESCRIPTION OF THE DRAWINGS

FIG. 1 shows a perspective view of a device according to a first exemplary embodiment of the present disclosure, being inserted through a working channel of an endoscope;

FIG. 2 shows another perspective view of the device of FIG. 1;

FIG. 3 shows a side view of the device of FIG. 1;

FIG. 4 shows a perspective view of a needle of the device of FIG. 1;

FIG. 5 shows a perspective view of the device of FIG. 1, in a first position;

FIG. 6 shows a perspective view of the device of FIG. 2, in a second position;

FIG. 7 shows a perspective view of a device according to a second exemplary embodiment of the present disclosure; and

FIG. 8 shows a perspective view of a suture of the device of FIG. 7.

### DETAILED DESCRIPTION

The present disclosure may be further understood with reference to the following description and the appended drawings, wherein like elements are referred to with the same reference numerals. The present disclosure related to devices for tissue closure and, in particular, to tissue closure devices inserted to target areas within a living body via a working channel of, for example, an endoscope or other insertion device. As would be understood by those skilled in the art, the device is flexible for insertion through a body lumen accessed, for example, via a naturally occurring body orifice.

Exemplary embodiments of the present disclosure describe a tissue closure device comprising a helical needle rotated about a longitudinal axis thereof such that a distal tip thereof alternatingly pierces portions of tissue extending along opposing sides of a tissue opening to hold the tissue opening in a closed configuration. In a further embodiment, the helical needle may be hollow such that a suture may be inserted therethrough. Once the suture has been passed through the needle, the needle may be withdrawn so that the suture remains in the tissue to hold the tissue opening in the closed configuration. It should be noted that the terms “proximal” and “distal” as used herein, are intended to refer to a direction toward (proximal) and away from (distal) a user of the device.

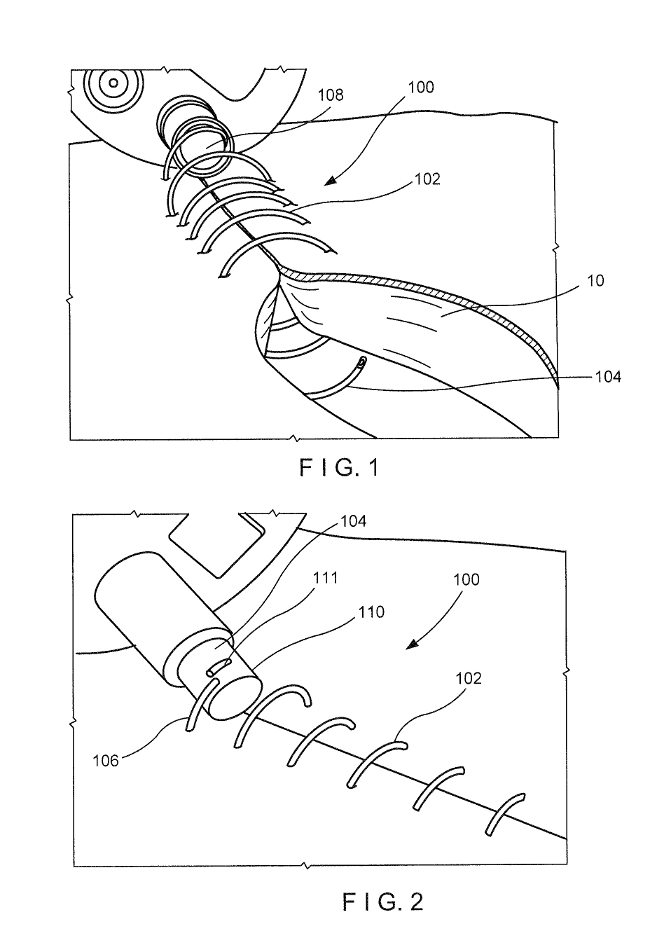

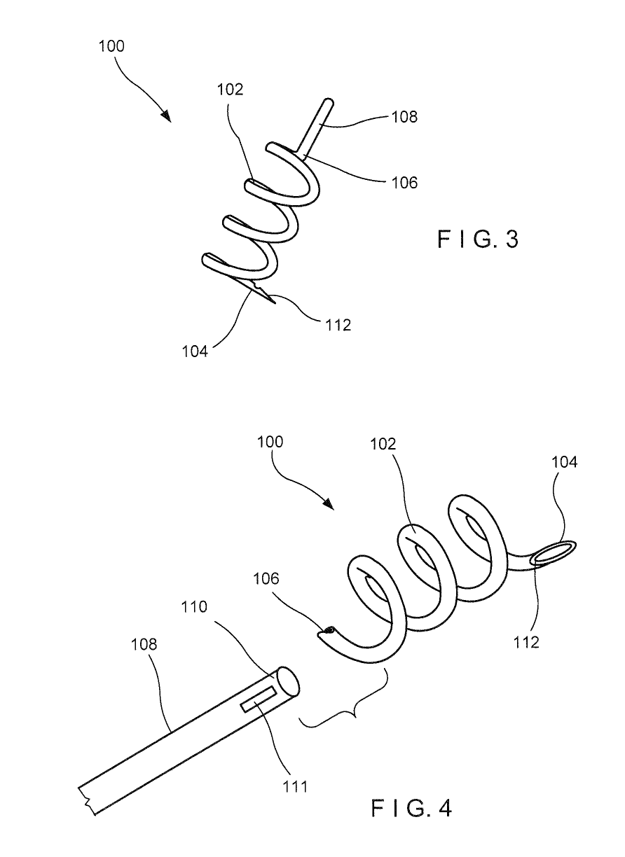

As shown in FIGS. 1-6, a tissue closure device 100 according to an exemplary embodiment of the present disclosure comprises a needle 102 extending about a longitudinal axis in a helical pattern from a distal end 104 to a proximal end 106 and a control member 108 coupled to the proximal end to deliver a torsional force thereto. The distal end 104 includes a tissue piercing distal tip 112 which may be tapered. As shown in FIGS. 1-2, the device 100 may be inserted to a target area within a living body via a working channel of, for example, an endoscope or other insertion device such as, for example, a laparoscope. The control member 108 may extend longitudinally from a distal end 110 coupled to the proximal end 106 of the needle 102 to a proximal end (not shown) extending to, for example, a handle assembly, which remains outside the body accessible to a user when the needle 102 is inserted to a target site within the body. The handle assembly may include an actuator for rotating the control member 108 to deliver the torsional force to the needle 102. After the needle 102 has been passed through the working channel to a target site, the needle 102 is rotated about the longitudinal axis via the control member 108 such that the distal tip 112 of the needle 102 extends distally past a distal end of the working channel and pierces a first portion of tissue along a first side 12 of a tissue opening 10. Upon piercing the first portion of tissue 12, the working channel (e.g., endoscope or laparoscope) may be moved toward a second portion of tissue along a second side 14 of the tissue opening 10 substantially opposite the first portion 12, thereby drawing the first portion of tissue 12 toward the second portion of tissue 14. The needle 102 may then be further rotated such that the distal tip 112 pierces the second portion of tissue 14. This process is repeated so that the distal tip 112 alternatingly pierces portions of tissue on the first and second sides 12, 14 of the tissue opening 10 until the needle 102 is wound about a length of the tissue opening 10 to hold the tissue opening 10 in a closed configuration.

In one exemplary embodiment, the needle 102 is preferably sized to fit into a working channel of an insertion instrument with which it is to be used. Alternatively, a diameter of the helix of the needle 102 may, when unconstrained, be larger than that of the working channel so that, when inserted, the diameter of the helical needle 102 is constrained by the interior surface of the working channel and, when the needle 102 is moved distally out of the working channel, it reverts under its natural bias to an expanded configuration. For example, the needle 102 may be formed of a superelastic shape memory material biased toward the expanded configuration and constrained by the working channel when inserted therein. In another embodiment, the helical shape of the needle 102 may not be uniform along a length thereof. In particular, a diameter of the helical shape of the needle 102 may vary along its length. For example, a diameter of the helical shape of the needle 102 may be larger at the distal end 104 of the needle 102 to aid in the piercing of tissue. In another example, a diameter of the helical shape of the needle 102 may be smaller at the distal and proximal ends 104, 106 so that, upon deployment of the needle 202, the needle 102 is held in place along the tissue opening 10. A length of the needle 102 may be selected to substantially correspond to a length of the tissue opening 10 that is intended to be treated. The needle 102 may also be formed of a bioabsorbable material so that the needle 102 dissolves or is absorbed by the body as the tissue opening 10 heals.

The distal end 110 of the control member 108 may be releasably coupled to the proximal end 106 of the needle 102. As shown in FIGS. 2 and 4, the distal end 110 may include a notch 111 in which a portion of the proximal end 106 of the needle 102 is received. The notch 111 and the proximal end 106 may be configured to prevent the needle 102 and the control member 108 from rotating with respect to one another. Once the needle 102 has been embedded in the target tissue as desired, the control member 108 may simply be drawn proximally relative to the needle 102 to disengage the control member 108 from the needle. In another embodiment, the control member 108 may be rotated in a direction opposite a direction in which the control member 108 is rotated to advance the needle 102 into the body. It will be understood by those of skill in the art, however, that the needle 102 and the control member 108 may be coupled to one another by any of a variety of coupling mechanisms so long as the needle 102 and the control member 108 are non-rotationally and releasably coupled to one another.

The control member 108 is configured to pass torque from the proximal end thereof to the needle 102 coupled to the distal end 110 thereof. Rotation of the control member 108 may be controlled via, for example, an actuator of a handle assembly coupled to the proximal end of the control member 108. Actuation of the device 100 rotates the control member 108 to advance the needle 102 distally relative to the working channel as it rotated about the longitudinal axis of the needle 102. The control member 108 may further include wire braids or coils to further enhance the torque-passing properties thereof.

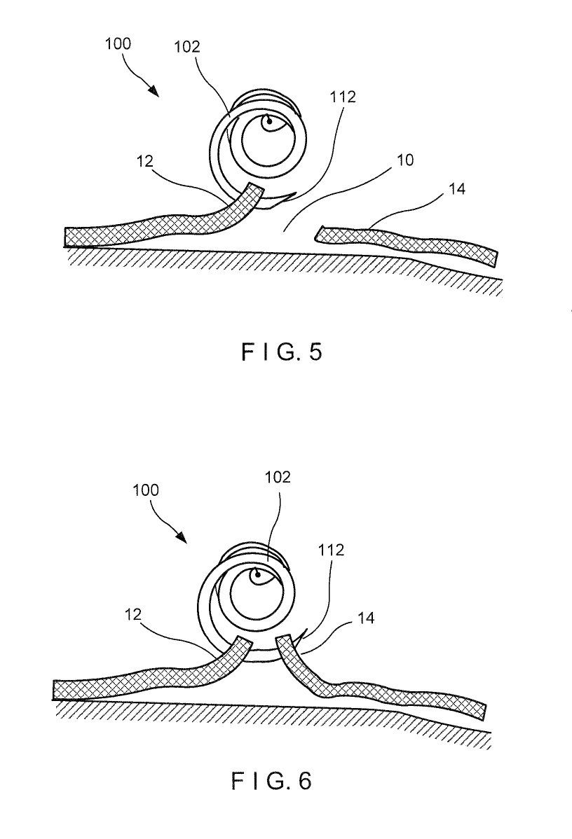

According to an exemplary surgical technique using the device 100, the needle 102 is inserted into a target area within a living body via the working channel of an insertion device. Although exemplary embodiments describe the insertion of the device 100 via an endoscope or other insertion device, it will be understood by those of skill in the art that the device 100 may be positioned in the patient body via any of a number of known methods. For example, the device 100 may be inserted through a guide, positioned manually or through an alternate or integrated steering device. Once the needle 102 has been positioned at the target area proximate the tissue opening 10, the control member 108 is rotated or advanced longitudinally to advance the needle 102 until the needle 102 extends distally out of the distal end of the working channel and the distal tip 112 pierces the first portion of tissue along the first side 12 of the tissue opening 10, as shown in FIG. 5. Opposing sides of the tissue opening 12, 14 may be approximated by moving the needle 102 (e.g., via the endoscope or laparoscope) toward the second side 14 of the tissue opening 10. The needle 102 may then be further rotated via the control member 108 so that the distal tip 112 pierces the second portion of tissue along the second side 14 of the tissue opening 10, as shown in FIG. 6. This process is repeated so that the distal tip 112 continues to pierce alternating sides 12, 14 of the tissue opening 10. A user of the device 100 may continue to move the needle 102 alternatingly toward an opposing side of the tissue opening 10 so that opposing edges of the tissue opening 10 are drawn toward one another as the needle 102 is extended therealong.

For example, once the second portion of tissue along the second side 14 of the tissue opening 10 has been pierced, the needle 102 may be moved toward the first side 12 of the tissue opening 10 so that the second portion of tissue is drawn toward the first side 12 of the tissue opening. The control member 108 is rotated so that the distal tip 112 of the needle 102 pierces a third portion of tissue along the first side 12 of the tissue opening 10, the third portion of tissue being distal of the first portion of tissue. The user continues to rotate the needle 102 in this manner until an entire length of the needle 102 has been embedded in tissue (e.g., wound about the tissue opening 10). It will be understood by those of skill in the art that the number of rotations required will depend on factors such as, for example, a size of the tissue opening, a number of turns of the helix of the needle 102, and the length of the needle 102. Once the needle 102 has been completely embedded in the tissue, the control member 108 may be disengaged from the needle 102 by, for example, drawing the control member 108 proximally with respect to the needle 102, so that only the needle 102 remains in the body.

Although the exemplary embodiments show and describe a single needle 102, the device 100 may include a plurality of needles 102, each of which is housed within the working channel axially aligned with one another, with the control member coupled to a proximal-most one of the needles 102. The needles 102 may be releasably coupled to one another so that rotation of the control member 108 is translated to a distal-most one of the needles 102. The needles 102 may be placed one at a time along the tissue opening 10, substantially as described above, until the tissue opening 10 is closed.

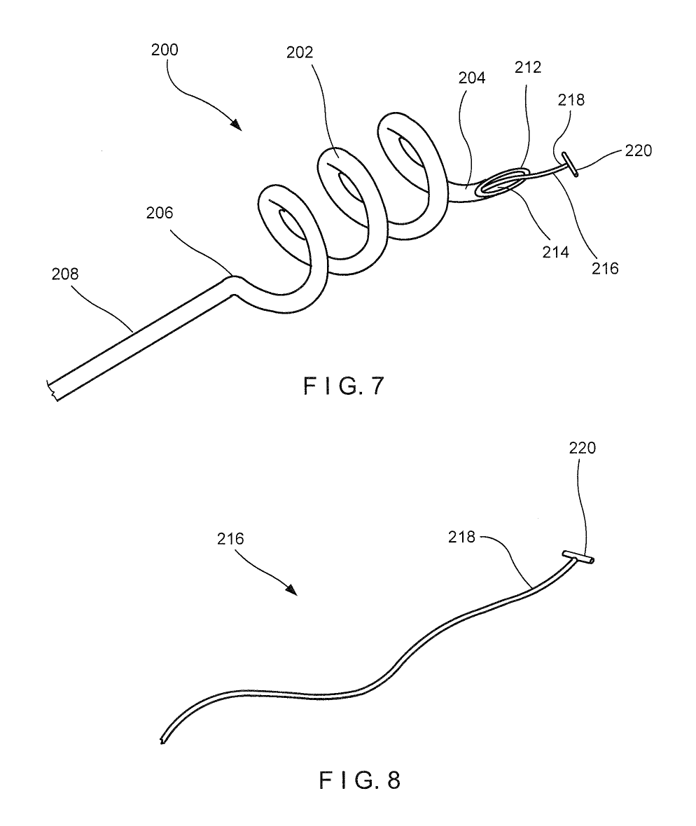

As shown in FIGS. 7-8, a device 200 according to a second exemplary embodiment of the present disclosure may be substantially similar to the device 100 described above, comprising a needle 202 extending about a longitudinal axis in a helical pattern and a control member 208 for delivering a torsional force to the needle 202. The needle 202 is substantially similar to the needle 102 extending from a proximal end 206 coupled to the control member 208 to a distal end 204 including a tissue piercing distal tip 212. The needle 202 includes a lumen 214 extending therethrough.

The device 200 further comprises a suture 216 slidably received in the lumen 214 so that, upon inserting the needle 202 into a patient body to close a tissue opening as described above in regard to the device 100, the suture 216 may be passed through the lumen 214 so that a distal end 218 of the suture extends distally past the distal end 204 of the needle 202 to engage a portion of tissue into which it is inserted. As the needle 102 is passed through the various portions of tissue around the opening to draw the opening closed, the suture 216 is also drawn through the tissue. When the needle 202 has reached a desired final position with the various portions of tissue surrounding the opening drawn together to seal the opening, the suture 216 is advanced distally out of the needle 202 to anchor an anchoring element 220 at the distal end of the suture within the tissue. The needle 202 may then be withdrawn from the body (e.g., by withdrawing the needle 202 proximally over the suture 216) leaving only the suture 216 threaded through alternating portions of tissue on first and second sides of the tissue opening to hold the opening closed. A proximal portion of the suture 216 may then be cinched, locked or tied to hold the suture 216 in place within the target tissue to maintain the tissue opening in the closed configuration.

The suture 216 may be formed of a polymer material such as, for example, Polypropylene Nylon and/or polyester, having sufficient column strength for the initial release of the anchoring element 220, and may be a monofilament or braided. The suture 216 may also be formed of a bioabsorbable material such that the suture 216 may dissolve or become absorbed by the body as the tissue opening heals. The suture 216 may be formed of hydrolytic degradable and/or absorbable materials such as, for example, Polyglycolic acid, Polyglactin based materials. The suture 216 may also have a surface lubrication coating and/or secondary composite to create less drag within the needle 202. Coatings may include materials such as, for example, Calcium stearate, glycol and poloxamers.

The anchoring element 220 is bent to extend longitudinally along the needle 202 when housed within the needle 202 and, when moved distally out of the needle 202, the anchoring element rotates under its natural bias to an anchoring position in which it extends transverse the needle 202. The anchoring element 220 according to this embodiment includes a laterally extending element such that the distal end 218 is substantially T-shaped. The laterally extending element of the anchoring element 220 may be deformed (e.g., bent) so that the suture 216 is easily slidable through the lumen 214. When the distal end 218 is pushed distally past the distal end of the lumen 214, however, the anchoring element 220 may revert to its T-shaped configuration to become anchored in the portion of tissue in which it is inserted. Although the anchoring element 220 is described and shown as a substantially T-shaped element, it will be understood by those of skill in the art that the anchoring element 220 may take any of a variety of shapes and configurations so long as the anchoring element 220 enables the distal end 218 of the suture 216 to become anchored in the tissue in which it is inserted. For example, the anchoring element 220 may also be a barb or a hook-shaped element. In another example, the anchoring element 220 may be formed as a helical structure biased toward a tighter spiral than the needle 202 so that, when the anchoring element 220 is moved distally past the distal end 204 of the needle 202, the anchoring element 220 reverts under its bias to the tighter spiral configuration to engage tissue into which it has been inserted.

The control member 208 may be integrally formed with the needle 202 such that the lumen 214 extends through both the needle 202 and the control member 208. The needle 202 and/or the control member 208 may be formed of, for example, a hypotube. As described above, the suture 216 is formed of a flexible material and is slidable through the lumen 214 however, the column strength of the suture 216 should be sufficient to enable the suture 216 to be pushed distally through the needle 202. The suture 216 extends from the distal end 218 to a proximal end (not shown) which, when the suture 216 is received within the lumen 214 extends proximally from a proximal end of the control member 208 so that it is accessible to a user of the device 200.

As described above, a portion of the suture 216 extending proximally from the tissue in which it is threaded may be tied in a knot to hold the suture 216 in place within the tissue. Alternatively, the device 200 may further comprise a locking element which may be positioned over the proximal portion of the suture 216 upon withdrawing the needle 202 therefrom. The locking element may, for example, include a cinch or clamp tightened over the proximal portion of the suture 216. The locking element may be housed within the working channel to be applied upon withdrawal of the needle 202. Alternatively, the locking element may be applied over the proximal portion of the suture 216 from within the needle 202. In another embodiment, the suture 216 may include a second anchoring element along a proximal portion thereof which, when the needle 202 is withdrawn, anchors the proximal portion of the suture 216 in the tissue

An exemplary surgical technique for closing a tissue opening using the device 200 may be substantially similar to the surgical technique described above in regard to the device 100. Similarly to the device 100, the device 200 may be inserted into a living body via a working channel of an insertion device and, once the needle 202 has been embedded in the target tissue adjacent to the tissue opening by rotating the needle 202 so that the distal end 214 alternatingly pierces portions of tissue along opposing sides of the tissue opening, the suture 216 is moved distally with respect to the needle 202 so that the distal end 218 of the suture 216 extends distally past the distal end 204 of the needle 202 and the anchoring element 220 lodges in the tissue. Where the anchoring element 220 is, for example, a T-shaped element, the anchoring element 220 reverts to the T-shape as it is pushed distally past the distal end 204 of the needle 202. Once the anchoring element 220 is anchored in the tissue, the needle 202 is withdrawn be rotating control member 208, and thereby the needle 202, in a reverse direction of which it was inserted. To further tighten the suture 216, the user may pull the proximal end of the suture 216 proximally relative to the working channel. Once the suture 216 has been positioned and/or tightened, as desired, the proximal portion of the suture 216 extending from the tissue may be tied in a knot or locked using the locking element so that the suture 216 remains threaded along the length of the tissue opening, holding the tissue opening in the closed configuration.

While embodiments have been described above, a number of modifications and changes may be made without departing from the scope of the disclosure. Thus, it is intended that the present disclosure cover modifications and variations provided that they come within the scope of the appended claims and their equivalents.

1-15. (canceled)

16. A device for treating tissue, comprising:
a helical needle extending about a longitudinal axis thereof from a distal tip to a proximal end;
a control member extending longitudinally between a distal end releasably coupled to the proximal end of the helical needle and a proximal end which, when the helical needle is inserted into a body, remains outside the body, the control member configured such that rotation of the control member about the longitudinal axis rotates the helical needle to screw the helical needle distally into a target tissue.

17. The device of claim 16, wherein a helical diameter of the helical needle varies along a length thereof.

18. The device of claim 16, wherein the helical needle is formed of a superelastic shape memory material.

19. The device of claim 16, wherein, when an unconstrained helical diameter of the helical needle is greater than an inner diameter of a working channel of an insertion instrument through which it is to be inserted to the target tissue within the body, the helical diameter is compressible via the working channel so that, when the helical needle is extended distally out of the working channel, the helical needle expands to the unconstrained helical diameter.

20. The device of claim 16, wherein the proximal end of the control member is connected to a handle assembly including an actuator for rotating the control member.

21. The device claim 16, wherein the control member and the helical needle are integrally formed.

22. The device of claim 16, wherein the helical needle includes a lumen extending therethrough.

23. The device of claim 22, further comprising a flexible suture slidably received within the lumen.

24. The device of claim 23, wherein a distal end of the flexible suture includes a first anchoring element configured to lodge in the target tissue into which it is inserted.

25. The device of claim 24, wherein the first anchoring element is one of T-shaped, a barb, a hook and a spiral.

26. The device of claim 24, wherein the first anchoring element is deformable into an insertion configuration in which it extends substantially parallel to a longitudinal axis of the flexible suture while the flexible suture resides within the helical needle and, when the distal end of the flexible suture is extended out of the distal end of the helical needle, the first anchoring element moves under a natural bias to an anchoring position transverse to the longitudinal axis of the flexible suture.

27. The device of claim 23, further comprising one of a locking element mountable over a portion of the flexible suture to lock the flexible suture within the body and a second anchoring element along a proximal portion of the flexible suture for anchoring the proximal portion of the flexible suture in a portion of the target tissue.

28. A system for treating a tissue, comprising:
a needle extending along a helical path about a longitudinal axis;
a control member extending longitudinally from a proximal end to a distal end releasably connected to the proximal end of the needle; and
an actuator coupled to the proximal end of the control member such that rotation of the control member rotates the needle and screws the needle into a target tissue.

29. The system of claim 28, further comprising a suture slidably received within a lumen of the needle, a distal end of the suture including an anchoring element so that when the distal end of the suture is moved distally past the distal end of the needle, the anchoring element lodges in the target tissue into which it is inserted.

30. The system of claim 28, wherein, when an unconstrained diameter of the helical path is greater than an inner diameter of a working channel through which it is to be inserted to the target tissue in the body, the needle being compressed when in the working channel so that, when the needle is moved distally out of the working channel, the needle expands to the unconstrained diameter of the helical path.

31. A method for treating a tissue opening, comprising:
inserting a helical needle through an insertion device to a tissue opening within a body, the helical needle extending along a helical path about a longitudinal axis to a tissue piercing distal end;
advancing distally a control member, a distal end of which is coupled to a proximal end of the helical needle, to advance the helical needle out of the insertion device into a first portion of tissue at a first side of the tissue opening, the helical needle being screwed into the first portion of tissue by rotation of the control member about the longitudinal axis;
moving the tissue opening toward a closed position by drawing the first portion of tissue toward a second side of the tissue opening opposite the first side of the tissue opening using the insertion device; and
rotating the control member to screw the helical needle into a second portion of tissue along the second side of the tissue opening to hold the tissue opening in the closed position.

32. The method of claim 31, further comprising:
drawing the second portion of tissue toward the first side of the tissue opening by moving the insertion device to move the helical needle toward the first side of the tissue opening; and
rotating the control member to screw the helical needle into a third portion of tissue along the first side of the tissue opening.

33. The method of claim 31, further comprising disengaging the control member from the helical needle so that the helical needle remains within the body to hold the tissue opening in the closed position.

34. The method of claim 31, further comprising inserting a suture through a lumen of the helical needle so that a distal end of the suture extends distally past the distal end of the helical needle to lodge in a portion of tissue into which it is inserted.

35. The method of claim 34, further comprising withdrawing the helical needle from the body by rotating the helical needle in a direction opposite the direction in which it was rotated during insertion into the target tissue so that the suture remains threaded through portions of tissue on the first and second sides of the tissue opening to hold the tissue opening in the closed position.
